(19) United States
(12) Patent Application Publication
(10) Pub. No.: US 2009/0240048 A1
(43) Pub. Date: Sep. 24, 2009

(54) Alpha-crystalline form of substituted selenoxanthenes and the method of its preparation

(75) Inventors: Rakhimdzhan A. ROZIEV, Obninsk (RU); Anatoly F. TSYB, Obninsk (RU); Anna Ya. GONCHAROVA, Obninsk (RU); Viktor V. KHOMICHONOK, Obninsk (RU); Vladimir K. PODGORODNICHENKO, Obninsk (RU)

Correspondence Address: HOUSTON ELISEEVA, 4 MILITIA DRIVE, SUITE 4, LEXINGTON, MA, 02421, US

(73) Assignee: MEDBIOPHARM LTD, Obninsk (RU)

(21) Appl. No.: 12/406,461

(22) Filed: Mar. 18, 2009

(30) Foreign Application Priority Data
Mar. 18, 2008 (RU) 2008109966

(51) Int. Cl. C07D 345/00

(52) U.S. Cl. 540/1

(57) ABSTRACT

The invention pertains to field of organic chemistry, medicine, pharmacology, foods and cosmetics industry, particularly, to manufacturing technology of selenoxanthenes; the invention may be used in manufacturing of food supplements, pharmaceutical and cosmetic products, having bioactive properties, of a wide spectrum of activity. The proposed compound is the α-crystalline form of 9-phenyl-symmetrical-octahydroselenoxanthene with exhibits antioxidant, detoxifying, immunomodulating, anti-atherogenic, anti-sclerotic, anabolic and hypolipidemic properties, and has the following structural formula:

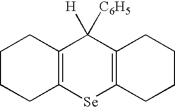

Its powder X-ray diffractogram (obtained from a Cu-K X-ray source) has characteristic diffractions in degrees of the diffraction angle 2theta as 6.0, 12.0, 15.0, 17.0, 19.0, 20.0, 21.5, 21.7, 20.9, 25.0, 27.0, 28.0, 29.0, 37.0; the melting point of 96.8° C. The crystalline form of the respective 9-R-symmetrical-selenoxantene product is obtained by crystallization from a weakly polar or a non-polar solvent.

### RELATED APPLICATIONS

This application claims priority to Russian Patent Application No. 2008109966, filed on Mar. 18, 2008, which is incorporated herein by reference in its entirety.

### FIELD OF THE INVENTION

This invention pertains to field of organic chemistry, medicine, pharmacology, foods and cosmetics industry, particularly, to manufacturing technology of selenoxanthenes; the invention may be used in manufacturing of food supplements, pharmaceutical and cosmetic products, having bioactive properties, of a wide spectrum of activity.

### BACKGROUND OF THE INVENTION

Known substitution derivatives of selenoxanthenes (RU 2213092, RU 2239632) have a following structural formula:

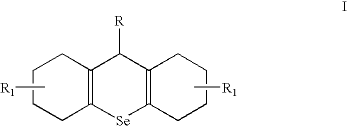

At this time, substitution derivatives of selenoxanthenes are manufactured in its amorphous form, the main disadvantage of which is its instability for storage; the compound has a low friability with tendency to clump.

Selenoxanthene and the method of its preparation are described in Patents RU 2213092, RU 2239632 and RU 2281007. These publications note that the product can recrystallize from ethanol or acetone.

Said compound's utilization and activity are described in the above-mentioned publications, specifically, in RU 2281007. According to this patent, 9-phenyl-symmetrical-octahydroselenoxanthene exhibits antioxidant, detoxifying, hypolipidemic, immunomodulating and immunocorrecting, anti-atherogenic, anti-atherosclerotic and anabolic properties. A report on toxicological properties of selenoxanthene (O.V. Meralenko, MVD, Ph.D. Laboratory of Bioactive Substances, Belgorod Agricultural Academy, 1996) demonstrates that substitution derivatives of selenoxanthene are highly toxic when administered intragastrically at LD50=725+75 mg/kg. The above indications limit the application of the substitution derivatives of selenoxanthene for food supplements and pharmaceutical compounds.

### SUMMARY OF THE INVENTION

The inventors' main objective was to obtain a non-toxic substitution derivative of selenoxanthene, with is stable for storage and has a high friability.

To achieve the above objective, proposed is a compound such as a selenoxanthene of α-crystalline form, with its powder X-ray diffractogram (obtained from a Cu-K X-ray source) having characteristic diffractions (in degrees of the diffraction angle 2theta) as follows: 6.0, 12.0, 15.0, 17.0, 19.0, 20.0, 21.5, 21.7, 20.9, 25.0, 27.0, 28.0, 29.0, 37.0, as shown below in FIG. 1.

The powder X-ray diffractogram was obtained using a D8 Advance diffractometer (Bruker) (T=298K, λCu-Kα X-ray source, Göbel (Goebel) mirrors, theta-2theta scan with 0.02+ step).

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 shows an X-ray diffractogram.

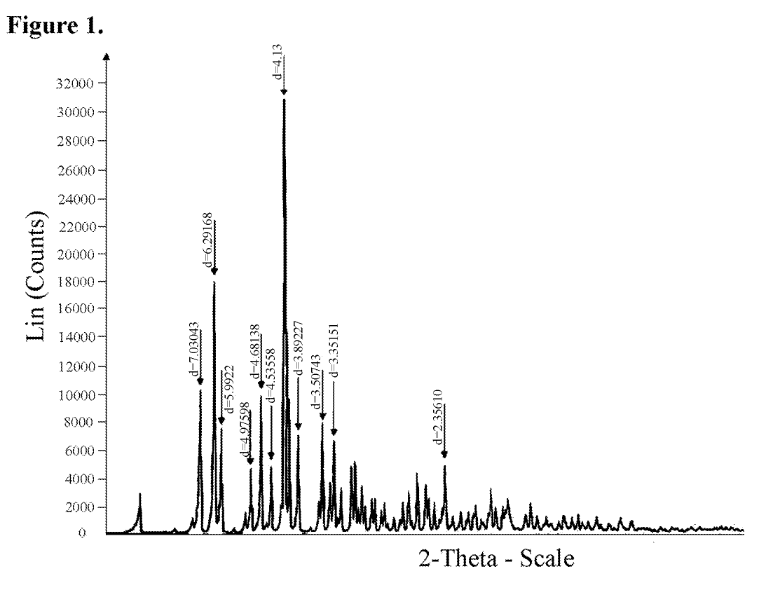

### DETAILED DESCRIPTION OF THE PREFERRED EMBODIMENTS

Experiment Description

X-ray structural analysis

Crystal of the compound H is tetragonal belonging to space group P-42₁c, at T=100K a=b=19.5515(15) Å, c=8.0074(6) Å, V=3060.9(4) Å³, Z(Z′)=8 (1), F(000)=1360, d_{calc}=1.429 g·sm⁻³, μ=24.42 mm⁻¹. Intensities of 12,199 diffractions were measured using an automatic diffractometer SMART APEX II CCD (Bruker) (T=100 K, λMo-Kα- X-ray source, graphite monochromator, (φ- and ω- scans, θₘₐₓ=58°), subsequent calculations were based on 4,063 independent diffractions (R(int)=0.0444). X-ray absorption calculations were done using the SADABS software package (G. M. Sheldrick, SADABS, V2.01, Bruker/Siemens Area Detector Absorption Correction Program; Bruker AXS Inc., Madison, Wis. 1998). The structure was determined via a direct method and full-matrix least-squares refinement in anisotropic approximation for non-hydrogen atoms. Positions of the hydrogen atoms were calculated geometrically and all of them were approximated by isotropic approximation with fixed positional and temperature parameters. Final divergence factors are: R₁=0.0306 for 3506 independent diffractions with I>2σ(I) and wR₂=0.0614, and GOF=0.999 for all independent reflections. All calculations were performed using SHELXTL PLUS (Version 5.10) software package (G. M. Sheldrick, SHELXTL, V5.10, Bruker AXS Inc., Madison, Wis, 1998).

TABLE 1
Crystal data and structure refinement for H.

| | |
|---|---|
| Identification code | H |
| Empirical formula | C19 H22 Se |
| Formula weight | 329.33 |
| Temperature | 100(2) K |
| Wavelength | 0.71073 Å |
| Crystal system | Tetragonal |
| Space group | P-42₁c |
| Unit cell dimensions | a = 19.5515(15) Å = 90°. |
| | b = 19.5515(15) Å = 90°. |
| | c = 8.0074(6) Å = 90°. |
| Volume | 3060.9(4) Å³ |
| Z | 8 |
| Density (calculated) | 1.429 Mg/m³ |
| Absorption coefficient | 2.442 mm⁻¹ |
| F(000) | 1360 |
| Crystal size | 0.45 × 0.40 × 0.40 mm³ |
| Theta range for data collection | 1.47 to 28.99°. |
| Index ranges | −21 <= h <= 26, −26 <= k <= |
| | 19, −10 <= 1 <= 8 |
| Reflections collected | 12199 |
| Independent reflections | 4063 [R(int) = 0.0444] |
| Completeness to theta = 28.99° | 99.9% |
| Absorption correction | Semi-empirical from equivalents |
| Max. and min. transmission | 0.377 and 0.340 |
| Refinement method | Full-matrix least-squares on F² |
| Data/restraints/parameters | 4063/0/182 |
| Goodness-of-fit on F² | 0.999 |
| Final R indices for 3506 refl. | R1 = 0.0306, wR2 = 0.0586 |
| with [I > 2sigma(I)] | |
| R indices (all data) | R1 = 0.0410, wR2 = 0.0614 |
| Absolute structure parameter | 0.007(8) |
| Extinction coefficient | 0.0022(2) |
| Largest diff. peak and hole | 0.438 and −0.334 e · Å⁻³ |

The production is obtained as follows. An amorphous powder (produced by a known technology) is crystallized either from a weak polar solvent selected from a group containing methanol, isopropanol, or from an aprotonic solvent selected from a group containing chloroform, hexane.

To prove the achievement of the technical result, the data assessing the proposed compound's acute toxicity are presented. The general toxicity study has shown no toxic effects following a single administration (intragastrically) to rats of the dosage of 1000 mg/kg of body mass of the anumal, which is more than a 1000 times greater that average therapeutic dose.

Physical and chemical data of the amorphous and crystalline forms are presented in the following table:

| | Angle of | Melting | Storage life, | |
|---|---|---|---|---|
| | repose, ° | point, ° C. | months | Appearance |
| Compound | 52° | 95-96° | 6 | Beige powder |
| according to | | | | |
| patent RU | | | | |
| 2281007 | | | | |
| (acetone) | | | | |
| Crystalline | 43° | 96.8° | 24 | Yellow needle- |
| form (hexane) | | | | like crystals |

Implementation of the method:

To produce 9-(o-oxyphenyl)-symmetrical-octahydroselenoxanthene, 12.5 g of 9-(o-oxyphenyl)-octahydro-10-oxoxanthene was placed in a three-neck flask with a magnetic stirrer and attached to a gas source. 30 ml of acetic acid:acetic anhydride (4:1) solution was then added. The reaction was stirred with nitrogen gas being blown through the liquid for 30 minutes; after 30 minutes nitrogen is replaced with hydrogen selenide. The rate of hydrogen selenide introduction is 2-3 bubbles per second. After initial 30 minutes of hydrogen selenide introduction, 1.0 ml of a concentrated hydrochloric acid was added to the reaction every hour. The total time of reaction in the presence of hydrogen selenide was 6 hours, after which the hydrogen selenide gas was replaced with the nitrogen gas for another 40 minutes. The reaction mixture was then refrigerated and after 24 hours the precipitate was filtrated and washed with hydrochloric acid and ethanol. The product yield was 11.5 g.

### EXAMPLE 1.

Re-crystallization was carried out as follows: 10 g of the amorphous product was placed in a 250 ml flask and 85 ml of hexane was added. The solution was brought to a boil and filtrated. After the crystals fell out, they were filtrated, washed with cold ethanol and dried at 40° C. The product yield was 8.7 g, the melting temperature of 96.8° C.

### EXAMPLE 2.

Re-crystallization was carried out as follows: 10 g of the amorphous product was placed in a 500 ml flask and 250 ml of isopropanol were added. The solution was brought to a boil and filtrated. After the crystals fell out, they were filtrated, washed with cold ethanol and dried at 40° C. The product yield was 9.2 g, the melting temperature of 96.6° C.

While this invention has been particularly shown and described with references to preferred embodiments thereof, it will be understood by those skilled in the art that various changes in form and details may be made therein without departing from the scope of the invention encompassed by the appended claims.

What is claimed is:

1. A compound comprising an α-crystalline form of 9-phenyl-symmetrical-octahydroselenoxanthene having a structural formula: 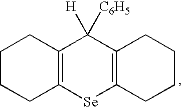
a melting point of 96.8° C., and
a powder X-ray diffractogram obtained from a Cu-K X-ray source having characteristic diffractions in degrees of the diffraction angle 2theta as 6.0, 12.0, 15.0, 17.0, 19.0, 20.0, 21.5, 21.7, 20.9, 25.0, 27.0, 28.0, 29.0, 37.0.

2. A method of producing a crystalline form of substituted 9-R-symmetrical-hydroselenoxantenes by crystallizing the 9-R-symmetrical-hydroselenoxantenes from a weakly polar or a non-polar solvent.

3. The method according to claim 2, wherein the weakly polar or non-polar solvent is selected from the group comprising hexane, chloroform, isopropanol.
